(19) United States
(12) Patent Application Publication
(10) Pub. No.: US 2026/0363565 A1
(43) Pub. Date: Nov. 19, 2026

(54) ADAPTIVE MULTI-MODAL BIOMETRIC SIGNAL PROCESSING AND CLOSED-LOOP RESPONSE CONTROL ARCHITECTURE

(71) Applicant: Jennifer Sabatini, Milwaukee, WI (US)

(72) Inventor: Jennifer Sabatini, Milwaukee, WI (US)

(73) Assignee: ABA Pro Software LLC, Lewes, DE (US)

(21) Appl. No.: 19/676,833

(22) Filed: May 14, 2026

Related U.S. Application Data
(60) Provisional application No. 63/807,445, filed on May 16, 2025.

(51) Int. Cl. A61B 5/024; A61B 5/00

(52) U.S. Cl. CPC A61B 5/02405; A61B 5/7264; A61B 5/7282

(57) ABSTRACT

An adaptive multi-modal biometric signal processing and closed-loop response control architecture acquires biometric sensor data and behavioral event marker data during a user session. A user-state computation engine establishes a session-specific calibration baseline, computes anomaly scores relative to the baseline, gates biometric feature values satisfying an outlier criterion, renormalizes weights for non-gated feature values, normalizes feature values, and applies signal type-specific weighted fusion to compute a unified user-state score classified against state profiles. A trained machine learning classifier generates an intervention output and assigns a posterior-probability confidence score. Low-confidence outputs are routed to a supervisory interface and withheld pending approval. An outcome measurement module computes a pre/post intervention outcome metric, and a parameter update module applies recency-weighted post-classifier probability multipliers to subsequent intervention selections during the session without updating trained model weights.

### CROSS-REFERENCE TO RELATED APPLICATIONS

This application claims the benefit under 35 U.S.C. § 119(e) of U.S. Provisional Patent Application No. 63/807,445, filed May 16, 2025, entitled “A Multi-Modular, Scalable System and Method Incorporating Real-Time Feedback Utilizing Biometric Signals, Behavioral Analytics, and Adaptive AI Intervention Within a Closed-Loop Human Support Architecture,” the disclosure of which is incorporated herein by reference. The inventor has also filed the following co-pending United States provisional patent applications relating to other subject matter and/or related technology areas: (1) U.S. Provisional Application No. 63/873,103, filed Aug. 29, 2025, entitled “Systems and Methods for Multi-Modal Biometric Consent Governance with Adaptive Safeguards, Trust Modulation, and Immutable Ledgering”; (2) U.S. Provisional Application No. 63/956,705, filed Jan. 9, 2026, entitled “Systems and Methods for Execution-Layer Authorization and Accountability of AI-Initiated Irreversible Actions”; (3) U.S. Provisional Application No. 63/965,333, filed Jan. 21, 2026, entitled “Pre-Inference Artificial Intelligence Safety Firewall for Youth-Classified Users”; (4) U.S. Provisional Application No. 63/984,838, filed Feb. 17, 2026, entitled “Systems and Methods for Deterministic AI Output Regulation and Compliance Enforcement”; (5) U.S. Provisional Application No. 63/998,995, filed Mar. 6, 2026, entitled “HIPAA-Compliant AI Diagnostic Assistant with Retrieval-Time Access Gates and Contextual De-Identification”; and (6) U.S. Provisional Application No. 63/999,424, filed Mar. 7, 2026, entitled “Systems and Methods for Governed Retrieval-Augmented Generation Across Heterogeneous Regulated Data Sources.” No claim of priority or benefit is made herein to these additional applications.

### FIELD OF THE INVENTION

The present invention relates to systems and methods for real-time biometric and behavioral monitoring with artificial intelligence-driven intervention selection, and more particularly to systems that compute a unified user-state representation from multi-modal biometric and behavioral data, generate confidence-scored intervention outputs through a trained machine learning classifier, deliver those outputs through role-differentiated interfaces, measure post-intervention outcomes, and automatically update intervention selection parameters through a closed-loop recency-weighted feedback architecture. The present invention is applicable across a broad range of deployment domains, including without limitation applied behavior analysis, occupational therapy, clinical psychology, workplace wellness, sports performance monitoring, elder care, and any context in which real-time physiological state monitoring and adaptive intervention selection are beneficial. In some embodiments, the system performs multi-modal signal processing, confidence evaluation, supervisory routing, and closed-loop parameter updating without requiring diagnosis, medical determination, psychological classification, or autonomous clinical decision-making.

### BACKGROUND OF THE INVENTION

### I. Limitations of Existing Biometric Monitoring Systems

Prior art biometric monitoring systems collect one or more physiological signals and generate alerts or reports based on static threshold comparisons. Prior art systems disclosed in the biometric monitoring literature monitor individual physiological signals such as heart rate variability (HRV) in isolation and compare measured values against fixed population-level thresholds. These systems suffer from three fundamental technical deficiencies that the present invention addresses. First, single-modality monitoring systems are susceptible to signal noise, sensor artifact, and inter-individual physiological variation. A single elevated HRV reading may reflect physical exertion, emotional stress, or sensor movement artifact, and no single-signal system can reliably distinguish among these causes in real time. The resulting high false-positive rate renders such systems impractical for continuous deployment in clinical and behavioral health contexts. Second, prior art systems employ static intervention protocols that do not adapt based on measured intervention outcomes. In applied behavior analysis (ABA), occupational therapy, and related behavioral health disciplines, practitioners have long recognized that the efficacy of any given behavioral intervention varies substantially across individuals, sessions, and environmental contexts. Static protocol systems cannot account for this variability and therefore deliver interventions of declining effectiveness over time as individual response patterns shift. Third, no prior art system integrates multi-modal biometric signal fusion, confidence-scored AI intervention selection, role-differentiated output interfaces, and closed-loop outcome-driven parameter updating within a single unified architecture. While individual components of such a system exist in isolation in the prior art, their integration produces technical improvements that are not achievable through the independent deployment of any individual component, as described in detail herein.

### II. Limitations of Prior Art AI-based Intervention Systems

Prior art AI-driven behavioral intervention systems that select interventions based on user state data have been proposed, but are not understood by Applicant to disclose: (a) a multi-modal biometric feature vector fusion pipeline incorporating anomaly detection and signal-type-specific weighting; (b) a confidence score assigned to each intervention output with supervisory alert routing when confidence falls below a configurable threshold; (c) role-differentiated output views providing clinician, supervisor, caregiver, trainer, and operator interfaces with distinct data fields; or (d) a recency-weighted sliding window parameter update mechanism that adjusts AI selection parameters within a session based on measured intervention outcomes. Prior art multi-modal EEG-based cognitive state classification systems are not understood by Applicant to disclose behavioral event markers, intervention selection or delivery, role-differentiated interfaces, outcome metric measurement, or closed-loop parameter updating. Prior art closed-loop cognitive state monitoring systems limited to passive monitoring are not understood by Applicant to disclose intervention generation, confidence scoring, or role-differentiated output delivery. The combination of technical elements disclosed in the present invention constitutes a novel and non-obvious advance over the prior art, individually and in combination, for the reasons set forth herein and as further distinguished during prosecution.

### III. Unmet Technical Need

There exists a long-felt and unmet technical need in the behavioral health, applied behavior analysis, clinical psychology, occupational therapy, and related fields for a system that: (1) continuously acquires and processes multi-modal biometric and behavioral data in real time; (2) applies signal-specific weighted fusion with anomaly detection to produce a reliable unified user-state representation; (3) selects interventions using a trained machine learning classifier with quantified confidence; (4) routes low-confidence outputs to supervisory personnel for human review while simultaneously enabling autonomous delivery of high-confidence interventions; (5) measures the effectiveness of each delivered intervention through pre-and post-intervention biometric and behavioral delta measurement; and (6) uses measured outcomes to automatically update intervention selection parameters, enabling the system to learn and improve within each session and across sessions. Applicant is not aware of any prior art system, individually or in combination, that satisfies all six of these technical requirements. The present invention satisfies all six requirements and provides technical improvements to biometric signal processing, AI intervention selection, and closed-loop adaptive control that are not achievable through any combination of prior art elements. In some implementations, the disclosed architecture may be implemented in a prototype clinical platform, demonstrating operability and utility of the system architecture when configured as described herein.

### SUMMARY OF THE INVENTION

The present invention provides, in a first aspect, a computer-implemented system comprising: one or more hardware processors; one or more non-transitory computer-readable media storing instructions; one or more biometric sensors configured to acquire physiological signals including at least two of heart rate variability (HRV), electrodermal activity (EDA), facial action unit data, and vocal prosody features; a behavioral tracking engine configured to acquire behavioral event markers; a user-state computation engine configured to apply a signal type-specific weighted fusion function to normalized biometric feature vectors and behavioral event markers to generate a unified user-state score and classify the unified user-state score against predefined stress recovery profiles to produce a current user-state representation stored as a UserStateRecord; an artificial intelligence decision engine comprising a trained machine learning classifier configured to receive the current user-state representation, select an intervention output, and assign a confidence score computed as the posterior probability output of the trained classifier; an adaptive output interface configured to present the intervention output through role-differentiated views including a clinician view, a supervisor view, a caregiver view, a trainer view, and an operator view, and to route the intervention output to a supervisory role view when the confidence score fails to satisfy a configurable threshold, which in some embodiments has a default value of 0.72; an outcome measurement module configured to compute an outcome metric by evaluating the change in at least one stress-indicative biometric measure and at least one behavioral performance measure between a pre-intervention baseline and a post-intervention measurement window; and a parameter update module configured to update intervention selection parameters of the artificial intelligence decision engine using a recency-weighted sliding window of outcome metrics. In some embodiments, the behavioral tracking engine 120 is implemented as an integral module of the system 100; in other embodiments, it is implemented as a separate software component providing behavioral event markers to the system 100 via a defined interface. The current user-state representation is stored as a UserStateRecord 131 as illustrated in FIG. 4. The present invention provides, in a second aspect, a computer-implemented method comprising: acquiring biometric data from at least two sensor modalities; establishing a session calibration baseline; applying z-score anomaly detection at the individual feature dimension level; normalizing biometric feature vectors and behavioral event markers to a common numerical scale; applying a signal type-specific weighted fusion function to generate a unified user-state score; classifying the unified user-state score against predefined stress recovery profiles; generating an intervention output using a trained machine learning classifier with posterior probability confidence scoring and configurable supervisory routing; presenting the intervention output through role-differentiated output views; acquiring post-intervention biometric and behavioral data; computing an outcome metric; and updating intervention selection parameters using a recency-weighted sliding window of stored outcome metrics. The present invention provides, in a third aspect, one or more non-transitory computer-readable media storing instructions that, when executed by one or more hardware processors, cause the processors to perform the operations of the second aspect. The present invention is not limited to any particular application domain.

### BRIEF DESCRIPTION OF THE DRAWINGS

FIG. 1 is a block diagram of the adaptive biometric and behavioral monitoring system (100), showing the seven principal modules: biometric input module (110), behavioral tracking engine (120), user-state computation engine (130), artificial intelligence decision engine (140), adaptive output interface (150), outcome measurement module (160), and parameter update module (170), and their interconnecting data flows.

FIG. 2 is a flowchart of the computer-implemented method (200), showing the sequential steps beginning with biometric and behavioral data acquisition through the closed-loop parameter update cycle, including the confidence threshold decision branch routing to the supervisor view (152) when confidence is below T_conf.

FIG. 3 is a block diagram of the biometric signal processing pipeline of the user-state computation engine (130), showing the stages of calibration, anomaly score and outlier-criterion processing (e.g., z-score processing), feature extraction, normalization, multi-modal fusion, and state profile classification that produce the UserStateRecord (131).

FIG. 4 is a data structure diagram of the UserStateRecord (131), InterventionRecord (141), and OutcomeMetricRecord (161), showing all field names, data types, and descriptions for each structured record generated by the system.

FIG. 5 is a block diagram of the adaptive output interface (150) and role-differentiated views (151-155), showing the clinician view (151), supervisor view (152), caregiver view (153), trainer view (154), and operator view (155) with role-based access controls enforced and confidence threshold routing active.

FIG. 6 is a block diagram of the closed-loop feedback architecture, showing the data flow among the artificial intelligence decision engine (140), adaptive output interface (150), outcome measurement module (160), and parameter update module (170) that constitutes the within-session adaptive control loop.

### DETAILED DESCRIPTION OF THE PREFERRED EMBODIMENTS

The following detailed description sets forth specific embodiments of the invention with sufficient detail to enable a person of ordinary skill in the art to make and use the invention without undue experimentation. The description refers to the accompanying drawings, in which like reference numerals designate like elements throughout.

### I. System Architecture Overview

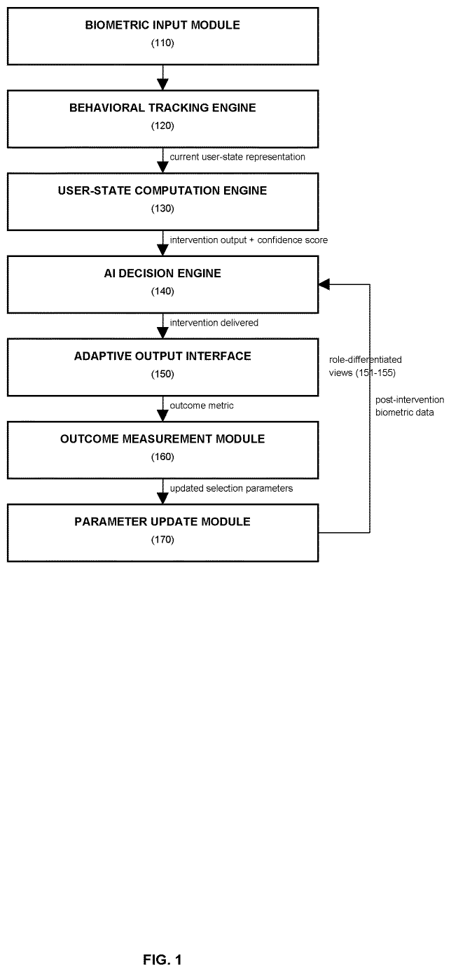

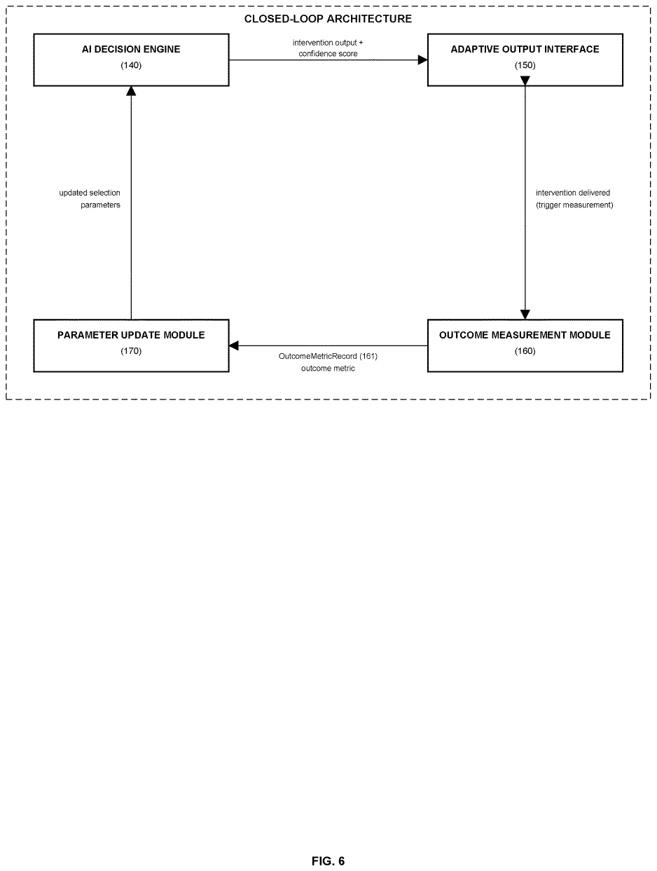

Referring to FIG. 1, the adaptive biometric and behavioral monitoring system 100 comprises seven principal modules interconnected by defined data flow paths. The biometric input module 110 and behavioral tracking engine 120 acquire raw data from the user and transmit processed data to the user-state computation engine 130. The user-state computation engine 130 generates a current user-state representation stored as a UserStateRecord 131 and transmits this representation to the artificial intelligence decision engine 140. The artificial intelligence decision engine 140 selects an intervention and assigns a confidence score, generating an InterventionRecord 141 and transmitting the intervention output and confidence score to the adaptive output interface 150. The adaptive output interface 150 delivers the intervention through role-differentiated views 151 through 155 and triggers outcome measurement. The outcome measurement module 160 receives post-intervention biometric data from the biometric input module 110 and post-intervention behavioral data from the behavioral tracking engine 120, computes an outcome metric, and generates an OutcomeMetricRecord 161. The parameter update module 170 receives the outcome metric from the outcome measurement module 160 and updates the intervention selection parameters of the artificial intelligence decision engine 140, completing the closed-loop feedback architecture illustrated in FIG. 6.

### II. Biometric Input Module (110)

The biometric input module 110 is configured to acquire physiological signals from one or more sensor modalities and to output biometric feature vectors for processing by the user-state computation engine 130. In preferred embodiments, the biometric input module 110 acquires signals from at least two of the following sensor modalities: (a) heart rate variability (HRV) sensors, including photoplethysmography (PPG) sensors, electrocardiography (ECG) sensors, and wearable optical pulse sensors; (b) electrodermal activity (EDA) sensors, including galvanic skin response (GSR) sensors and skin conductance sensors embedded in wearable devices; (c) facial action unit detection systems, including camera-based facial analysis modules configured to detect the presence and intensity of facial muscle movements associated with emotional and stress states as defined in the Facial Action Coding System (FACS); and (d) vocal prosody analysis modules configured to extract pitch, intensity, rate, and spectral features from audio inputs. The biometric input module 110 is sensor-agnostic with respect to hardware implementation. Suitable wearable sensors include, without limitation, wrist-worn biometric bands, chest-worn ECG patches, camera-based facial analysis systems, and microphone-based vocal analysis systems. The biometric input module 110 continuously acquires sensor data at a sampling rate sufficient to detect physiological state changes within a temporal resolution of two seconds or less. The architecture is extensible to additional physiological, behavioral, environmental, contextual, machine-state, or future sensor modalities without modification to the closed-loop adaptive control architecture. The biometric input module 110 also transmits post-intervention biometric data to the outcome measurement module 160 following delivery of each intervention by the adaptive output interface 150.

### III. Behavioral Tracking Engine (120)

The behavioral tracking engine 120 is configured to acquire behavioral event markers from one or more behavioral data sources and to transmit behavioral event data to the user-state computation engine 130 and post-intervention behavioral data to the outcome measurement module 160. Behavioral event markers acquired by the behavioral tracking engine 120 include, without limitation: (a) task engagement metrics, including time-on-task, task initiation latency, and task completion rate; (b) interaction event data, including input device activity, application interaction frequency, and session navigation patterns; (c) vocal interaction data, including speech rate, response latency, and verbal output volume; (d) physical movement data, including accelerometer-derived activity levels, postural indicators, and proximity sensor outputs; and (e) session timing data, including session duration, break frequency, and transition timing. The behavioral tracking engine 120 is software-implemented and may operate on one or more computing devices including wearable processors, mobile computing devices, workstation computers, and cloud-based processing infrastructure. The behavioral tracking engine 120 generates timestamped behavioral event marker records at a temporal resolution sufficient to detect behavioral state changes within a five-second window.

### IV. User-State Computation Engine (130)

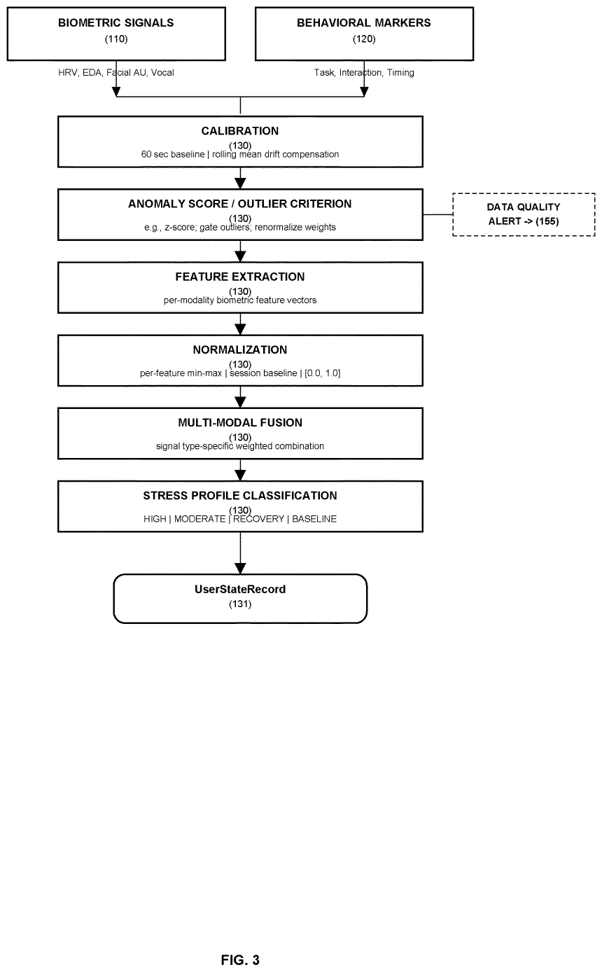

The user-state computation engine 130 receives biometric feature vectors from the biometric input module 110 and behavioral event markers from the behavioral tracking engine 120, and generates a current user-state representation stored as a UserStateRecord 131. The user-state computation engine 130 executes the biometric signal processing pipeline illustrated in FIG. 3.

### A. Signal Calibration

Upon session initialization, the user-state computation engine 130 establishes a baseline calibration measurement by acquiring biometric and behavioral data during a defined resting period of not less than sixty seconds. The calibration module applies baseline drift compensation by computing a rolling mean of each biometric signal over a configurable window of not less than thirty seconds and subtracting the rolling mean from current sensor readings to produce baseline-compensated feature values. This calibration step addresses the technical problem of inter-session and intra-session sensor baseline drift, which causes false-positive stress detections in prior art single-threshold systems.

### B. Anomaly Detection

Following calibration, the user-state computation engine 130 applies an anomaly detection module to each incoming biometric feature vector. The anomaly detection module computes an anomaly score for each biometric feature value relative to the session-specific calibration baseline and determines whether the feature value satisfies an outlier criterion. In some embodiments, the anomaly score is a z-score and the outlier criterion is satisfied when the z-score exceeds a configurable outlier threshold, including without limitation 3.0 standard deviations. Feature values satisfying the outlier criterion are gated by exclusion from a subsequent weighted fusion computation, and one or more weights associated with remaining non-gated feature values may be renormalized for the subsequent multi-modal fusion. The corresponding anomaly flag field of the UserStateRecord 131 is set to TRUE for each affected feature dimension. When gated biometric feature values occur in more than a configurable fraction of active biometric sensor modalities during a common measurement interval, the user-state computation engine 130 generates a data quality alert transmitted to the operator view 155 or another supervisory or operator interface of the adaptive output interface 150.

### C. Feature Extraction and Normalization

The user-state computation engine 130 extracts a feature vector from each active sensor modality. HRV features include, without limitation, SDNN (standard deviation of normal-to-normal intervals), RMSSD (root mean square of successive differences), and LF/HF ratio (low-frequency to high-frequency spectral power ratio). EDA features include mean skin conductance level, skin conductance response rate, and peak amplitude. Facial features include action unit intensities for units AU1, AU2, AU4, AU6, AU7, AU12, AU17, and AU24 as defined in the Facial Action Coding System (FACS). Vocal features include mean fundamental frequency (F0), F0 standard deviation, speech rate in syllables per second, and intensity in decibels. Following feature extraction, the user-state computation engine 130 normalizes all extracted feature values to a common numerical scale in the range [0.0, 1.0] using a per-feature min-max normalization computed against the session calibration baseline. Normalization ensures that signal type-specific weighted fusion produces a unified score that is not dominated by features with larger absolute magnitudes.

### D. Multi-Modal Fusion and Stress Profile Classification

The user-state computation engine 130 applies a signal type-specific weighted fusion function to normalized non-gated biometric feature values and normalized behavioral feature values to produce a unified user-state score. The weighted fusion function is defined in some embodiments as: unified_score=sum(w_i multiplied by f_i) divided by sum(w_i), where f_i denotes a normalized feature value for the i-th active feature dimension and w_i denotes a signal type-specific or modality-dependent weight for the i-th feature dimension. When one or more feature dimensions or modalities are gated due to anomaly processing, the remaining weights may be rescaled so that a sum of weights applied in the fusion computation remains constant, equals 1.0, or otherwise satisfies a defined normalization condition. Signal type-specific weights reflect the relative diagnostic reliability of each biometric and behavioral signal type in a relevant configuration and may be determined by cross-validated training on domain-specific labeled datasets. In preferred embodiments for behavioral health configurations including applied behavior analysis (ABA), occupational therapy, and clinical psychology, HRV features are assigned a weight coefficient in the range [0.25, 0.40], EDA features are assigned a weight coefficient in the range [0.20, 0.35], facial action unit features are assigned a weight coefficient in the range [0.15, 0.30], and vocal features are assigned a weight coefficient in the range [0.10, 0.25], with the sum of all active weight coefficients normalized to 1.0. The unified user-state score is then classified against predefined state profiles. In preferred embodiments, the predefined state profiles include stress recovery profiles comprising four classes: (1) HIGH stress, corresponding to unified scores in the range [0.75, 1.00]; (2) MODERATE stress, corresponding to unified scores in the range [0.50, 0.75); (3) RECOVERY, corresponding to unified scores in the range [0.25, 0.50); and (4) BASELINE, corresponding to unified scores in the range [0.00, 0.25). The state profile boundaries are configurable by authorized users with appropriate privileges.

### E. Userstaterecord Generation

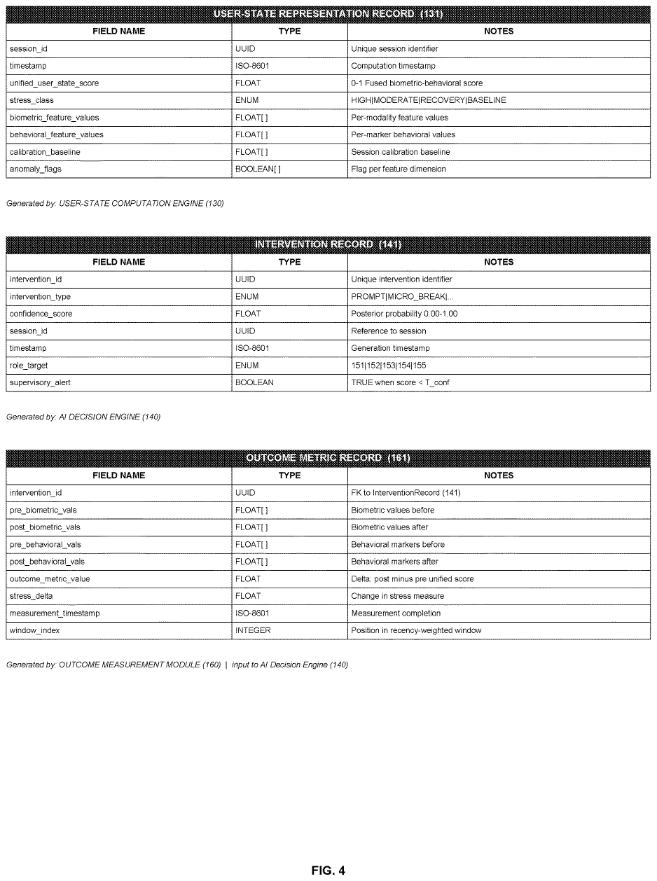

The user-state computation engine 130 stores the results of each computation cycle as a UserStateRecord 131 comprising the following fields: session_id (UUID); timestamp (ISO-8601 datetime); unified_user_state_score (FLOAT, range [0.0, 1.0]); stress_class (ENUM: HIGH, MODERATE, RECOVERY, BASELINE); biometric_feature_values (FLOAT array, one value per active feature dimension); behavioral_feature_values (FLOAT array, one value per active behavioral event marker dimension); calibration_baseline (FLOAT array); and anomaly_flags (BOOLEAN array, one value per active feature dimension). The UserStateRecord 131 structure is illustrated in FIG. 4.

### F. Terminology and Configurable Signal-Processing Embodiments

As used herein, an anomaly score comprises any computed measure of deviation, inconsistency, or abnormality of a biometric feature value relative to a session-specific calibration baseline, and may include, without limitation, a z-score, a robust z-score, a percentile deviation score, an interquartile-range score, a Mahalanobis-distance score, a model-based residual score, a probabilistic outlier score, a sensor-quality score, or combinations thereof. An outlier criterion comprises any rule, threshold, range test, quality-control condition, or model output used to determine whether a biometric feature value is treated as anomalous for purposes of downstream processing. In some embodiments, biometric feature values satisfying the outlier criterion are gated by exclusion from subsequent multi-modal fusion.

In embodiments in which one or more biometric feature values are gated from fusion, the system may renormalize weights associated with non-gated feature values such that fusion is performed using weights corresponding to active, non-gated features and/or modalities. For example, when one or more feature dimensions or modalities are excluded due to anomaly gating, the remaining weights may be rescaled so that a sum of weights applied in the fusion computation remains constant, equals 1.0, or otherwise satisfies a defined normalization condition.

As used herein, state profiles comprise predefined classifications of the unified user-state score and/or related derived measures, and may include, without limitation, stress recovery profiles, arousal profiles, recovery profiles, behavioral readiness profiles, workload profiles, engagement profiles, fatigue profiles, or other predefined state classifications.

As used herein, a supervisory interface comprises any role-differentiated interface configured to receive low-confidence intervention outputs for human review, approval, modification, or rejection prior to delivery, and may be implemented as a supervisor view within a role-based access controlled user interface, an alerting console, or an integrated external governance service.

In some embodiments, within-session adaptation is achieved by updating session-specific intervention selection parameters applied after classifier inference, such as post-classifier probability multipliers, pacing constraints, and/or confidence thresholds. The post-classifier probability multipliers may comprise scalar multipliers applied to posterior probabilities or scores associated with respective intervention classes. Such updates may be performed without retraining the trained machine learning classifier and without updating trained model weights, thereby enabling within-session adaptation using engineered control parameters rather than model-weight updates.

### V. Artificial Intelligence Decision Engine (140)

The artificial intelligence decision engine 140 receives the UserStateRecord 131 from the user-state computation engine 130 and generates an intervention output comprising a selected intervention type and a confidence score. The artificial intelligence decision engine 140 comprises a trained machine learning classifier configured to receive the current user-state representation as an input feature vector and produce a probability distribution over available intervention classes as output.

### A. Classifier Architecture

In preferred embodiments, the trained machine learning classifier is implemented as a gradient-boosted ensemble classifier trained on a labeled dataset of user-state representations paired with expert-validated intervention selections and measured outcomes. The classifier receives as input features the unified_user_state_score, stress_class encoded as a one-hot vector, biometric_feature_values, behavioral_feature_values, session elapsed time, and a configurable set of historical intervention outcome features derived from the recency-weighted sliding window of outcome metrics maintained by the parameter update module 170. The artificial intelligence decision engine 140 is inference-engine agnostic. Alternative embodiments may implement the classifier as a random forest classifier, a support vector machine with a radial basis function kernel, a multilayer perceptron neural network, a long short-term memory (LSTM) recurrent neural network, or a transformer-based sequence model. The selection of classifier architecture does not affect the scope of the claims.

### B. Confidence Score Computation

The artificial intelligence decision engine 140 assigns a confidence score to each generated intervention output. The confidence score is computed as the posterior probability of a selected intervention class as output by the trained classifier, expressed as a floating-point value in the range [0.00, 1.00]. For a classifier producing a softmax output layer, the confidence score for the selected intervention class k is defined as: confidence_score=exp(z_k)/sum(exp(z_j)) for all j in intervention_classes where z_k denotes the logit output for class k. This formulation ensures that the confidence score reflects genuine classifier uncertainty across the full intervention class distribution.

### C. Supervisory Alert Routing

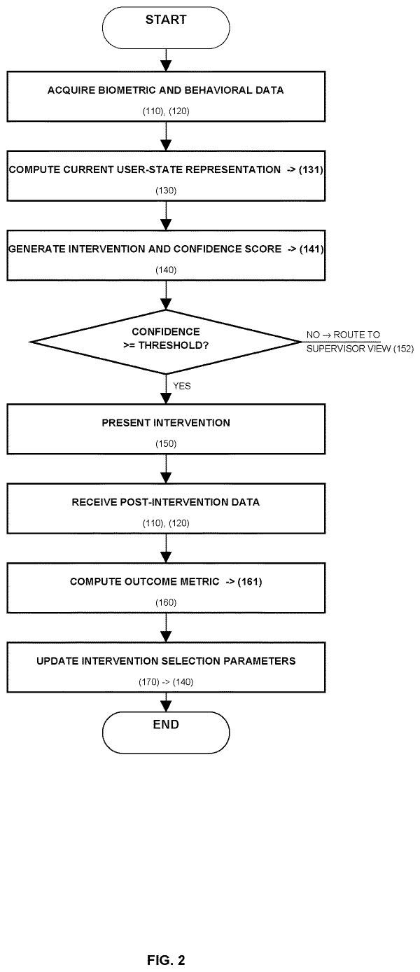

The artificial intelligence decision engine 140 compares the confidence score against a configurable confidence threshold T_conf having a default value of 0.72. When the confidence score is greater than or equal to T_conf, the intervention output is routed to the adaptive output interface 150 for autonomous delivery to the appropriate role-differentiated view. When the confidence score is less than T_conf, the intervention output is routed to the supervisor view 152 with a supervisory alert flag set to TRUE in the InterventionRecord 141, indicating that the intervention requires human review and approval before delivery. This supervisory routing mechanism enables the system to maintain clinician oversight while enabling autonomous intervention delivery for high-confidence outputs, as illustrated in FIG. 2.

### D. Interventionrecord Generation

The artificial intelligence decision engine 140 stores each generated intervention output as an InterventionRecord 141 comprising the following fields: intervention_id (UUID); intervention_type (ENUM: PROMPT, MICRO_BREAK, DE-ESCALATION, REDIRECT, PACING_CUE, SENSORY_MODULATION); confidence_score (FLOAT, range [0.00, 1.00]); session_id (UUID); timestamp (ISO-8601 datetime); role_target (ENUM: 151, 152, 153, 154, 155); and supervisory_alert (BOOLEAN, TRUE when confidence_score<T_conf). The InterventionRecord 141 structure is illustrated in FIG. 4.

### E. Bias Mitigation

The artificial intelligence decision engine 140 incorporates a bias mitigation module that evaluates the distribution of intervention types generated across demographic subgroups defined within the user population. The bias mitigation module computes an intervention distribution parity metric by comparing the frequency of each intervention type across subgroups and generates a bias alert when the parity metric exceeds a configurable disparity threshold, in preferred embodiments set to a maximum frequency ratio of 2.0. The bias mitigation module transmits bias alerts to the supervisor view 152 for human review.

### VI. Adaptive Output Interface (150)

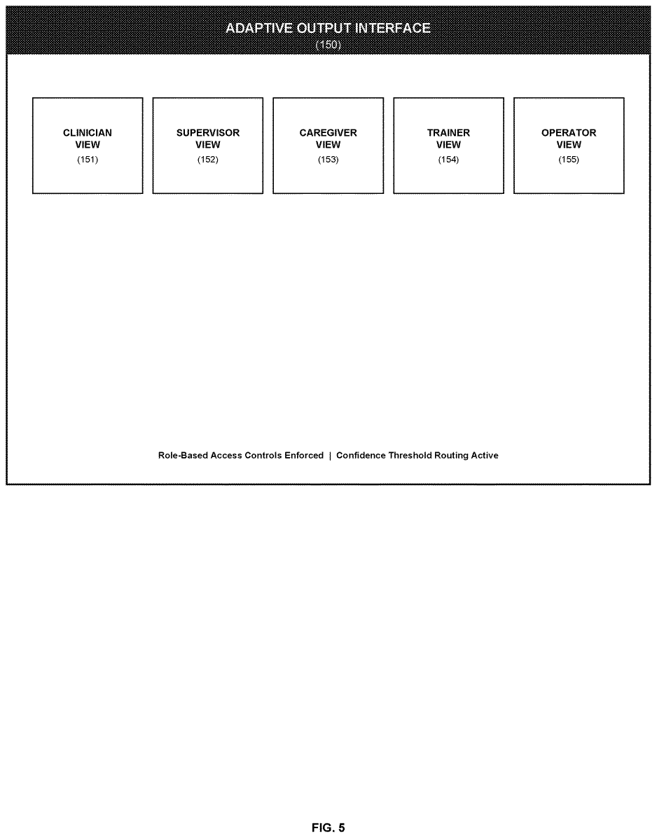

The adaptive output interface 150 receives intervention outputs and confidence scores from the artificial intelligence decision engine 140 and delivers interventions to authorized users through role-differentiated views enforced by a role-based access control (RBAC) schema. The adaptive output interface 150 is illustrated in FIG. 5.

### A. Role-based Access Control Architecture

The RBAC schema of the adaptive output interface 150 defines five primary roles: Clinician (151), Supervisor (152), Caregiver (153), Trainer (154), and Operator (155). Each role is associated with a defined set of data access permissions, intervention delivery permissions, and alert routing rules. Role assignments are administered by authorized system administrators and are enforced at the application layer through cryptographically signed session tokens. All role assignments and permission changes are recorded in an immutable access log.

### B. Clinician View (151)

The clinician view 151 provides authorized clinical personnel with access to: the full text and type of each delivered intervention; the confidence score associated with each intervention; user-state history charts displaying the unified_user_state_score and stress_class over the session timeline; biometric feature trend visualizations; and the intervention delivery history for the current session and configurable historical periods.

### C. Supervisor View (152)

The supervisor view 152 provides authorized supervisory personnel with access to: supervisory alerts generated when confidence scores fall below the configurable threshold T_conf; intervention approval and rejection controls; oversight activity logs recording all supervisory actions with timestamps; and bias alert notifications. The supervisor view 152 is the primary interface through which human oversight is maintained over AI-generated intervention outputs.

### D. Caregiver View (153)

The caregiver view 153 provides authorized caregiving personnel with access to: daily and session-level progress summaries displaying stress profile distributions and intervention frequency; de-escalation alerts indicating when the user-state computation engine 130 has classified the current user state as HIGH stress; and simplified trend reports suitable for communication with families, guardians, and support networks.

### E. Trainer View (154)

The trainer view 154 provides authorized training and coaching personnel with access to: session pacing visualizations displaying the temporal distribution of stress profile classifications; intervention timeline charts; performance indicator dashboards displaying task engagement metrics, completion rates, and behavioral event marker trends; and intervention frequency metrics.

### F. Operator View (155)

The operator view 155 provides authorized system operations personnel with access to: system health monitoring dashboards; sensor connectivity status indicators for all active biometric sensor modalities; data pipeline diagnostic tools including signal quality indicators, calibration status displays, and anomaly detection frequency reports; and system configuration interfaces enabling authorized operators to modify configurable parameters including the confidence threshold T_conf, anomaly detection thresholds, and normalization parameters.

### G. Output Modalities

The adaptive output interface 150 delivers intervention outputs through one or more output modalities including: visual display interfaces on wearable devices, mobile computing devices, and workstation displays; haptic feedback actuators providing vibrotactile intervention cues; audio output interfaces providing voice-synthesized intervention prompts; and application programming interfaces enabling integration with electronic health record (EHR) systems and behavior management platforms.

In some embodiments, intervention authorization, supervisory escalation routing, audit logging, role-based access enforcement, and/or execution control are implemented through an external governance, authorization, or policy-enforcement framework interfaced with the adaptive output interface (150) and/or the artificial intelligence decision engine (140). In such embodiments, the external framework may operate as a separate service or process and may enforce organization-specific policies without requiring modification to the biometric input module (110), the user-state computation engine (130), or the parameter update module (170).

### VII. Outcome Measurement Module (160)

The outcome measurement module 160 is configured to compute a quantitative outcome metric for each delivered intervention by comparing pre-intervention and post-intervention biometric and behavioral measurements. The outcome measurement module 160 is triggered by the delivery of an intervention by the adaptive output interface 150.

### A. Pre-intervention Baseline Capture

Upon receiving a trigger from the adaptive output interface 150, the outcome measurement module 160 captures the pre-intervention baseline as the UserStateRecord 131 most recently generated by the user-state computation engine 130 prior to intervention delivery, specifically the unified_user_state_score, stress_class, biometric_feature_values, and behavioral_feature_values fields of that record.

### B. Post-intervention Measurement Window

The outcome measurement module 160 acquires post-intervention biometric data from the biometric input module 110 and post-intervention behavioral data from the behavioral tracking engine 120 over a configurable post-intervention measurement window. In preferred embodiments, the post-intervention measurement window begins two seconds after intervention delivery and extends for a configurable duration of not less than thirty seconds and not more than three hundred seconds. The two-second start delay excludes the immediate physiological startle response from the outcome metric computation.

### C. Outcome Metric Computation

The outcome measurement module 160 computes the outcome metric as the difference between the post-intervention unified_user_state_score and the pre-intervention unified_user_state_score: outcome_metric =post_unified_score-pre_unified_score A negative outcome_metric indicates stress reduction following the intervention. A positive outcome_metric indicates stress increase following the intervention. The outcome_metric is a floating-point value in the range [−1.0, 1.0].

### D. Outcomemetricrecord Generation

The outcome measurement module 160 stores each computed outcome metric as an OutcomeMetricRecord 161 comprising the following fields: intervention_id (UUID); pre_biometric_vals (FLOAT array); post_biometric_vals (FLOAT array); pre_behavioral_vals (FLOAT array); post_behavioral_vals (FLOAT array); outcome_metric_value (FLOAT, range [−1.0, 1.0]); stress_delta (FLOAT); measurement_timestamp (ISO-8601 datetime); and window_index (INTEGER). The OutcomeMetricRecord 161 structure is illustrated in FIG. 4.

### VIII. Parameter Update Module (170)

The parameter update module 170 is configured to receive OutcomeMetricRecords 161 from the outcome measurement module 160 and to update the intervention selection parameters of the artificial intelligence decision engine 140 based on the measured outcomes of previously delivered interventions. The parameter update module 170 implements the closed-loop adaptive control architecture illustrated in FIG. 6.

### A. Recency-weighted Sliding Window

The parameter update module 170 maintains a sliding window of the N most recent OutcomeMetricRecords 161 for the current session, where N is a configurable window size with a default value of 20 records. Each record in the sliding window is assigned a recency weight computed as: recency_weight(i)=exp(−lambda*(N−i)) where i is the index of the record within the window (i=N for the most recent record) and lambda (lambda) is a configurable decay parameter with a default value of 0.10. This exponential recency weighting ensures that more recent intervention outcomes exert greater influence on parameter updates than older outcomes.

### B. Parameter Update Computation

The parameter update module 170 computes an adjustment delta for each adjustable intervention selection parameter based on the recency-weighted average outcome metric across intervention types or intervention classes. The adjustable parameters include: (a) intervention type priority weights, which may be implemented as post-classifier probability multipliers applied to classifier output probabilities for each intervention type; (b) confidence threshold T_conf; (c) intervention pacing parameters including minimum inter-intervention intervals; and (d) session-specific feature weights in the weighted fusion function of the user-state computation engine 130. In some embodiments, for each intervention class, the parameter update module 170 computes a recency-weighted mean outcome metric associated with previously delivered intervention outputs of that intervention class and adjusts a corresponding post-classifier probability multiplier for the intervention class based on the recency-weighted mean outcome metric. The parameter adjustment delta for an intervention type priority weight w_k may be computed as: delta_w_k=learning_rate multiplied by recency_weighted_mean_outcome(intervention_type_k), where learning_rate is a configurable parameter with a default value of 0.05. Updated priority weights or probability multipliers may be clipped to a defined range, including without limitation [0.10, 2.00], to prevent parameter divergence.

### C. Feedback to Artificial Intelligence Decision Engine

Following each parameter update computation, the parameter update module 170 transmits the updated intervention selection parameters to the artificial intelligence decision engine 140. The artificial intelligence decision engine 140 applies the updated parameters to all subsequent intervention selections within the session. This within-session closed-loop adaptation enables the system to improve intervention efficacy over the course of a session without requiring retraining of the underlying machine learning model. The updated intervention selection parameters are implemented as post-classifier probability multipliers applied to classifier output probabilities, adjustments to the configurable confidence threshold T_conf, and modifications to intervention pacing constraints, constituting an engineered adaptive control architecture that is distinct from machine learning model retraining.

### IX. Alternative Embodiments

### A. Applied Behavior Analysis (ABA) Embodiment

In a preferred embodiment for applied behavior analysis (ABA) deployment, the system 100 may be implemented in a prototype ABA clinical platform. The system 100 is configured to monitor individuals receiving ABA therapy in clinical, school, or home settings. The biometric input module 110 acquires HRV and EDA data from a wrist-worn wearable sensor and facial action unit data from a tablet-mounted camera. The behavioral tracking engine 120 acquires task engagement and completion data from an ABA data collection application executing on the tablet. The artificial intelligence decision engine 140 may be trained on a labeled dataset of ABA session recordings annotated by Board Certified Behavior Analysts (BCBAs). Intervention types delivered through the adaptive output interface 150 include discrete trial prompts, preference-based redirects, sensory modulation cues, and de-escalation protocols. The supervisor view 152 generates alerts when the confidence score falls below T_conf=0.72. In prototype implementation, the z-score anomaly detection pipeline of the present invention may reduce false-positive stress alerts that would otherwise result from sensor motion artifacts and individual baseline variability when static population-level thresholds are applied, consistent with the technical problem identified in the Background of the Invention. The within-session closed-loop parameter updating may improve the relevance of intervention timing across the course of individual sessions as individual response patterns are captured by the recency-weighted sliding window. This ABA embodiment is illustrative only and does not limit the invention to ABA therapy or clinical deployments.

### B. Occupational Therapy Embodiment

In an alternative embodiment for occupational therapy deployment, the system 100 is configured to monitor individuals during occupational therapy sessions. The biometric input module 110 acquires HRV data from a chest-worn ECG patch and EDA data from a palm-worn galvanic skin response sensor. The behavioral tracking engine 120 acquires motor task performance data from sensor-equipped therapy tools. Intervention types include pacing cues, activity modification prompts, sensory integration exercises, and rest breaks.

### C. Workplace Wellness Embodiment

In an alternative embodiment for workplace wellness deployment, the system 100 is configured to monitor employees in high-stress work environments. The biometric input module 110 acquires HRV data from a wrist-worn smartwatch and vocal prosody data from a desktop or headset microphone. The behavioral tracking engine 120 acquires keyboard and mouse interaction metrics, application engagement data, and communication frequency data from workplace productivity platforms. Intervention types delivered through the adaptive output interface 150 include micro-break prompts, breathing exercise cues, task prioritization redirects, workload adjustment recommendations, and focus mode activation cues. The within-session closed-loop parameter updating architecture adapts intervention timing and type to individual employee response patterns within each work session without requiring offline model retraining.

### D. Clinical Psychology Embodiment

In an alternative embodiment for clinical psychology deployment, the system 100 is configured to monitor patients during exposure therapy, cognitive behavioral therapy, and other structured clinical interventions. The confidence threshold T_conf is set to 0.85 for clinical deployments to maximize supervisory oversight. All interventions in clinical psychology embodiments are routed through the supervisor view 152 for psychologist approval before delivery.

### X. Security and Data Privacy

### A. Data Encryption

All biometric and behavioral data acquired, processed, transmitted, and stored by the system 100 is encrypted using AES-256 encryption. Data at rest is encrypted using AES-256 block cipher encryption. Data in transit is encrypted using Transport Layer Security (TLS) version 1.3 or higher. Encryption keys are managed using a hardware security module (HSM) or a cloud-based key management service (KMS) validated or configured to operate consistent with FIPS 140-2 Level 2 or higher.

### B. Access Logging

All access to user data, including read operations, write operations, role assignments, and configuration changes, is recorded in an immutable append-only access log. The access log records the user identifier, role, timestamp, operation type, and data record identifier for each access event. The access log is stored in a tamper-evident storage system and is accessible only to authorized system administrators through the operator view 155.

### C. Regulatory Support and Privacy Controls

The system 100 may be configured to support technical, administrative, and data-processing controls consistent with one or more regulatory frameworks, including the Health Insurance Portability and Accountability Act (HIPAA) Security Rule (45 C.F.R. Parts 160 and 164) for deployments involving protected health information; the Family Educational Rights and Privacy Act (FERPA) for deployments in educational settings; the General Data Protection Regulation (GDPR) for deployments within the European Union; the Illinois Biometric Information Privacy Act (BIPA); and the California Consumer Privacy Act (CCPA).

### D. De-Identification

The system 100 provides a configurable de-identification mode in which all user-identifying data fields are replaced with pseudonymous identifiers prior to transmission to external systems, cloud storage, or third-party analytics platforms, in accordance with the HIPAA Safe Harbor de-identification standard (45 C.F.R. § 164.514(b)).

### XI. Pseudocode Implementation

The following pseudocode illustrates the core processing loop of the system 100 and corresponds to the method steps illustrated in FIG. 2:
PROCEDURE AdaptiveBiometricMonitoringLoop(session_id):
   INITIALIZE calibration_baseline=AcquireCalibrationBaseline(duration=60 s)
   INITIALIZE sliding_window=EmptySlidingWindow(size=N)
   INITIALIZE selection_parameters=DefaultSelectionParameters( )
LOOP:
   biometric_data=BiometricInputModule.Acquire( )//FIG. 1: (110)
   behavioral_data=BehavioralTrackingEngine.Acquire( )//FIG. 1: (120)
   calibrated=Calibrate(biometric_data, calibration_baseline) validated, anomaly_flags, active_weights=GateAndRenormalize(calibrated, calibration_baseline, outlier_criterion)//FIG. 3
   feature_vectors=ExtractFeatures(validated)//FIG. 3
   normalized=Normalize(feature_vectors, calibration_baseline)//FIG. 3
unified_score=WeightedFusion(normalized, active_weights)//FIG. 3
state_profile=ClassifyStateProfile(unified_score)//FIG. 3
user_state_rec=CreateUserStateRecord(session_id, unified_score,
state_profile, feature_vectors, anomaly_flags)//->(131) FIG. 4
   intervention_type, confidence_score=AIDecisionEngine.SelectIntervention(user_state_rec, selection_parameters)// FIG. 1: (140)
   supervisory_alert=(confidence_score <T_conf)// FIG. 2: decision
   intervention_rec=CreateInterventionRecord(session_id, intervention_type, confidence_score, supervisory_alert)//->(141) FIG. 4
   IF confidence_score>=T_conf THEN
      AdaptiveOutputInterface. DeliverIntervention(intervention_rec, role_target)//FIG. 1: (150), FIG. 5
   ELSE
      AdaptiveOutputInterface. GenerateSupervisoryAlert(intervention_rec, supervisor_view=152)//FIG. 2: NO branch, FIG. 5
      approval=SupervisorView. AwaitApproval(intervention_rec)
      IF approval==APPROVED THEN
         AdaptiveOutputInterface. DeliverIntervention(intervention_rec, role_target)
      END IF
   END IF
   pre_state=user_state_rec
   WAIT post_intervention_window
   post_biometric=BiometricInputModule.Acquire( )
   post_behavioral=BehavioralTrackingEngine.Acquire( )
   post_score=ComputePostInterventionScore(post_biometric, post_behavioral)
   outcome_metric=post_score−pre_state.unified_score outcome_rec=
CreateOutcomeMetricRecord(intervention_rec. intervention_id, pre_state, post_biometric, post_behavioral, outcome_metric)//->(161) FIG. 4
   sliding_window.Append(outcome_rec)
   recency_weights=ComputeRecencyWeights(sliding_window, lambda=0.10)
selection_parameters=UpdatePostClassifierMultipliers(sliding_window, recency_weights, learning_rate=0.05)//FIG. 1: (170), FIG. 6

AIDecisionEngine.ApplyPostClassifierMultipliers(selection_parameters, without_model_weight_updates=True)//FIG. 6:->(140)
END LOOP
END PROCEDURE

### XII. Patent Eligibility Under 35 U.S.C. § 101

The present invention is directed to specific technical improvements to multi-modal biometric sensor data processing and adaptive machine learning-based intervention systems, and is directed to patent-eligible subject matter under 35 U.S.C. § 101. The following disclosure sets forth the specific technical improvements provided by the claimed invention for the benefit of examination under the USPTO January 2019 Revised Guidance and October 2019 Update.

### A. Technical Improvements to Biometric Sensor Data Processing

The claimed invention provides a specific technical improvement to the way biometric monitoring computing systems process multi-modal physiological sensor data and adapt intervention-selection control parameters during a user session. Prior art biometric monitoring systems applied static, population-level threshold comparisons to raw sensor signals from individual modalities, without session-specific calibration, without feature-level anomaly scoring and gating, without weight renormalization after exclusion of anomalous feature values, without signal type-specific weighted fusion of active non-gated feature values, and without within-session post-classifier probability multiplier updating. The present invention addresses these concrete technical deficiencies through an ordered, hardware-executed processing pipeline. Technical Improvement 1—Session-Specific Calibration Baseline: the system establishes a session-specific calibration baseline using biometric sensor data and behavioral event marker data acquired during an initialization period. Technical Improvement 2—Feature-Level Anomaly Scoring, Outlier Gating, and Weight Renormalization: the system computes anomaly scores relative to the session-specific calibration baseline, gates feature values satisfying an outlier criterion from subsequent multi-modal fusion, and renormalizes one or more weights associated with remaining non-gated feature values. Technical Improvement 3—Per-Feature Session-Relative Normalization: the system normalizes non-gated biometric feature values and behavioral feature values using per-feature normalization computed relative to the session-specific calibration baseline. Technical Improvement 4—Signal Type-Specific Weighted Multi-Modal Fusion: the system computes a unified user-state score by applying a signal type-specific weighted fusion function to normalized non-gated biometric feature values and normalized behavioral feature values. Technical Improvement 5—Within-Session Post-Classifier Parameter Updating Without Model-Weight Updates: the system updates session-specific intervention selection parameters, including post-classifier probability multipliers, during the user session for subsequent intervention selections without updating trained model weights of the trained machine learning classifier.

### B. Absence of Preemption

The claims do not preempt all approaches to biometric monitoring, anomaly detection in physiological data, multi-modal sensor fusion, or AI-driven intervention selection. A practitioner who omits the session-specific calibration baseline, who does not compute anomaly scores relative to that baseline, who does not gate outlier feature values from subsequent multi-modal fusion, who does not renormalize weights associated with non-gated feature values after gating, who applies equal-weight rather than signal type-specific weighted fusion, who omits confidence-based supervisory withholding, or who updates a trained machine learning classifier by retraining or updating trained model weights rather than applying within-session post-classifier probability multipliers does not practice the independent claims as drafted.

### C. Relationship to Judicial Precedent on Specific Technical Improvements

The specific technical improvements of the present invention are structurally and functionally analogous to physiological signal processing improvements recognized as patent-eligible in Cardionet, LLC v. Infobionic, Inc., 955 F.3d 1358 (Fed. Cir. 2020); McRO, Inc. v. Bandai Namco Games Am., Inc., 837 F.3 d 1299 (Fed. Cir. 2016); Enfish, LLC v. Microsoft Corp., 822 F. 3d 1327 (Fed. Cir. 2016); and BASCOM Global Internet Services, Inc. v. AT&T Mobility LLC, 827 F.3d 1341 (Fed. Cir. 2016). In each of those decisions, the Federal Circuit held patent-eligible claims directed to specific technical improvements to computing systems rather than abstract mathematical concepts implemented generically. The present claims are directed to a specific improvement to biometric monitoring computing systems implemented through a defined five-stage signal processing pipeline that produces qualitatively different structured outputs not achievable by prior art systems.

### D. Non-Obviousness Under 35 U.s.c. § 103

The claimed invention would not have been obvious to a person of ordinary skill in the art at the time of filing under 35 U.S.C. § 103. Distinction 1—Feature-Level Anomaly Scoring, Gating, and Weight Renormalization: Applicant is not aware of any prior art reference that discloses computing an anomaly score for each biometric feature value relative to a session-specific calibration baseline, excluding feature values satisfying an outlier criterion from a weighted multi-modal fusion computation, and renormalizing one or more weights associated with non-gated feature values for the subsequent multi-modal fusion. Distinction 2—Within-Session Recency-Weighted Parameter Updating Without Model-Weight Updates: Applicant is not aware of any prior art reference that discloses updating session-specific intervention selection parameters during a user session using a recency-weighted sliding window of outcome metrics, applying the updated parameters as post-classifier probability multipliers to subsequent intervention selections, and doing so without updating trained model weights of the trained machine learning classifier. Distinction 3—Confidence-Based Supervisory Withholding: Applicant is not aware of any prior art reference that discloses routing an AI-generated intervention output to a supervisory interface and withholding autonomous delivery of the intervention output pending receipt of supervisory approval when a posterior probability confidence score fails to satisfy a configurable threshold. Distinction 4—Per-Intervention-Class Feedback Updating: Applicant is not aware of any prior art reference that discloses computing, for each intervention class, a recency-weighted mean outcome metric associated with previously delivered intervention outputs of that intervention class and adjusting a corresponding post-classifier probability multiplier for that intervention class based on the recency-weighted mean outcome metric.

### E. Declaration of Technical Novelty and Non-conventional Character

Applicant submits, based on a review of the prior art known to Applicant as of the filing date and based on the technical disclosure set forth herein, that the following specific combination of technical elements was not well-understood, routine, or conventional in the art of biometric monitoring, physiological signal processing, or AI-driven behavioral intervention at the time of filing: (1) session-specific calibration using biometric sensor data and behavioral event marker data acquired during an initialization period; (2) baseline drift compensation applied to biometric signals; (3) anomaly score computation for biometric feature values relative to the session-specific calibration baseline; (4) gating of feature values satisfying an outlier criterion from subsequent multi-modal fusion; (5) renormalization of one or more weights associated with non-gated feature values for the subsequent multi-modal fusion; (6) per-feature normalization computed relative to the session-specific calibration baseline; (7) signal type-specific weighted fusion of normalized non-gated biometric feature values and normalized behavioral feature values to compute a unified user-state score; (8) trained machine learning classifier intervention selection with posterior probability confidence scoring and configurable supervisory withholding; (9) pre-and post-intervention outcome metric computation; and (10) within-session recency-weighted updating of session-specific intervention selection parameters applied as post-classifier probability multipliers to subsequent intervention selections without updating trained model weights. The Examiner is respectfully requested that any finding that the claimed technical combination was well-understood, routine, or conventional be supported by citations to specific prior art references that expressly disclose this combination, consistent with the evidentiary standard applicable to such findings under controlling Federal Circuit precedent.

### XIII. Alternative Embodiment—Reduced Pipeline Configuration

In an alternative embodiment, the system 100 may be deployed with a reduced sensor modality configuration comprising as few as two active biometric sensor modalities. In such reduced-configuration deployments, the anomaly detection module computes anomaly scores for available feature dimensions, gates feature values satisfying an outlier criterion from subsequent multi-modal fusion, and renormalizes signal type-specific weights for active non-gated features or modalities so that the sum of active weights equals 1.0 or otherwise satisfies a defined normalization condition.

What is claimed is:

1. A computer-implemented system that improves the technical accuracy of multi-modal biometric signal fusion and enables within-session adaptive intervention control by excluding sensor-artifact-contaminated feature values from fusion computations and adjusting intervention selection parameters based on measured intervention outcomes without retraining a machine learning model, the system comprising: one or more hardware processors;
and one or more non-transitory computer-readable media storing instructions that, when executed by the one or more hardware processors, cause the system to perform operations comprising:
receiving, during a user session, (i) biometric sensor data from at least two biometric sensor modalities and (ii) behavioral event marker data;
establishing a session-specific calibration baseline using biometric sensor data and behavioral event marker data acquired during an initialization period of the user session;
generating biometric feature values from the biometric sensor data and behavioral feature values from the behavioral event marker data;
for each of a plurality of biometric feature dimensions, computing an anomaly score relative to the session-specific calibration baseline, determining whether a biometric feature value satisfies an outlier criterion, and, when the outlier criterion is satisfied, improving fusion accuracy by gating the biometric feature value through exclusion from subsequent multi-modal fusion and renormalizing one or more weights associated with non-gated feature values so that the multi-modal fusion computation operates over a dynamically adjusted, artifact-free feature set;
normalizing non-gated biometric feature values and behavioral feature values using per-feature normalization computed relative to the session-specific calibration baseline;
computing a unified user-state score by applying a signal type-specific weighted fusion function to the normalized non-gated biometric feature values and the normalized behavioral feature values;
generating a current user-state representation by classifying the unified user-state score against predefined state profiles;
generating an intervention output using a trained machine learning classifier based at least on the current user-state representation, and assigning to the intervention output a confidence score comprising a posterior probability of a selected intervention class output by the trained machine learning classifier;
when the confidence score is less than a configurable confidence threshold, technically constraining system output by routing the intervention output to a supervisory interface and withholding autonomous transmission of the intervention output to any non-supervisory interface pending receipt of supervisory approval, thereby enforcing human oversight as a structural system constraint rather than a post-hoc process step;
computing an outcome metric associated with the intervention output by comparing (i) a pre-intervention unified user-state score to (ii) a post-intervention unified user-state score measured over a post-intervention measurement window; and
updating, during the user session and without retraining the trained machine learning classifier or modifying trained model weights, one or more session-specific intervention selection parameters applied as post-classifier probability multipliers to subsequent intervention selections, wherein the post-classifier probability multipliers are computed from a recency-weighted sliding window of outcome metrics that assigns greater influence to more recently measured intervention outcomes, thereby enabling within-session adaptive control of intervention selection through engineered parameter adjustment distinct from machine learning model training.

2. A computer-implemented method for improving the technical accuracy of multi-modal biometric signal fusion and enabling within-session adaptive intervention control, the method comprising:
receiving, during a user session, (i) biometric sensor data from at least two biometric sensor modalities and (ii) behavioral event marker data;
establishing a session-specific calibration baseline using biometric sensor data and behavioral event marker data acquired during an initialization period of the user session;
generating biometric feature values from the biometric sensor data and behavioral feature values from the behavioral event marker data;
for each of a plurality of biometric feature dimensions, computing an anomaly score relative to the session-specific calibration baseline, determining whether a biometric feature value satisfies an outlier criterion, and, when the outlier criterion is satisfied, improving fusion accuracy by gating the biometric feature value through exclusion from subsequent multi-modal fusion and renormalizing one or more weights associated with non-gated feature values so that the multi-modal fusion computation operates over a dynamically adjusted, artifact-free feature set;
normalizing non-gated biometric feature values and behavioral feature values using per-feature normalization computed relative to the session-specific calibration baseline;
computing a unified user-state score by applying a signal type-specific weighted fusion function to the normalized non-gated biometric feature values and the normalized behavioral feature values;
generating a current user-state representation by classifying the unified user-state score against predefined state profiles;
generating an intervention output using a trained machine learning classifier based at least on the current user-state representation, and assigning to the intervention output a confidence score comprising a posterior probability of a selected intervention class output by the trained machine learning classifier;
when the confidence score is less than a configurable confidence threshold, technically constraining system output by routing the intervention output to a supervisory interface and withholding autonomous transmission of the intervention output to any non-supervisory interface pending receipt of supervisory approval, thereby enforcing human oversight as a structural system constraint rather than a post-hoc process step;
computing an outcome metric associated with the intervention output by comparing (i) a pre-intervention unified user-state score to (ii) a post-intervention unified user-state score measured over a post-intervention measurement window; and
updating, during the user session and without retraining the trained machine learning classifier or modifying trained model weights, one or more session-specific intervention selection parameters applied as post-classifier probability multipliers to subsequent intervention selections, wherein the post-classifier probability multipliers are computed from a recency-weighted sliding window of outcome metrics that assigns greater influence to more recently measured intervention outcomes, thereby enabling within-session adaptive control of intervention selection through engineered parameter adjustment distinct from machine learning model training.

3. One or more non-transitory computer-readable media storing instructions that, when executed by one or more hardware processors, cause the one or more hardware processors to perform the method of claim 2.

4. The system of claim 1, wherein the at least two biometric sensor modalities are selected from the group consisting of heart rate variability (HRV) sensors, electrodermal activity (EDA) sensors, facial action unit detection systems, and vocal prosody analysis modules.

5. The system of claim 1, wherein the behavioral event marker data comprises at least two of task engagement metrics, interaction event data, physical movement indicators, vocal interaction markers, and session timing data.

6. The system of claim 1, wherein establishing the session-specific calibration baseline comprises acquiring at least sixty seconds of the biometric sensor data and the behavioral event marker data during the initialization period.

7. The system of claim 1, further comprising applying baseline drift compensation by computing a rolling statistic over a configurable window for at least one biometric signal and compensating current biometric measurements using the rolling statistic.

8. The system of claim 1, wherein computing the anomaly score comprises computing a z-score per biometric feature dimension relative to the session-specific calibration baseline.

9. The system of claim 8, wherein the outlier criterion is satisfied when an absolute value of the z-score exceeds a configurable threshold within a range of 2.0 to 4.0 standard deviations.

10. The system of claim 1, further comprising generating a data quality alert when gated biometric feature values occur in more than a configurable fraction of active biometric sensor modalities during a common measurement interval, and routing the data quality alert to an operator interface.

11. The system of claim 1, wherein normalizing the non-gated biometric feature values and the behavioral feature values comprises performing per-feature min-max normalization computed relative to the session-specific calibration baseline to map the non-gated biometric feature values and the behavioral feature values into a common numerical range.

12. The system of claim 1, wherein applying the signal type-specific weighted fusion function comprises computing the unified user-state score according to: unified_score=sum(w_i multiplied by f_i) divided by sum(w_i), in which f_i comprises a normalized feature value and w_i comprises a modality-dependent weight stored in memory.

13. The system of claim 12, wherein, in a behavioral-health configuration, HRV feature dimensions have weights in a range of 0.25 to 0.40, EDA feature dimensions have weights in a range of 0.20 to 0.35, facial action unit feature dimensions have weights in a range of 0.15 to 0.30, and vocal prosody feature dimensions have weights in a range of 0.10 to 0.25.

14. The system of claim 1, wherein the predefined state profiles comprise stress recovery profiles including at least HIGH, MODERATE, RECOVERY, and BASELINE.

15. The system of claim 1, wherein the posterior probability is computed from a softmax output of the trained machine learning classifier for the selected intervention class.

16. The system of claim 1, wherein withholding autonomous delivery of the intervention output comprises preventing transmission of the intervention output to a non-supervisory interface until the supervisory approval is received.

17. The system of claim 1, wherein the post-intervention measurement window begins not less than two seconds after delivery of the intervention output and has a duration between thirty seconds and three hundred seconds.

18. The system of claim 1, wherein the recency-weighted sliding window comprises N most recent outcome metrics for the user session, and wherein each outcome metric of the N most recent outcome metrics is weighted by an exponential decay function that assigns greater weight to more recently measured outcomes.

19. The system of claim 1, wherein updating the one or more session-specific intervention selection parameters comprises: computing, for each intervention class, a recency-weighted mean outcome metric associated with previously delivered intervention outputs of the intervention class;
and adjusting a corresponding post-classifier probability multiplier for the intervention class based on the recency-weighted mean outcome metric.

20. The system of claim 1, further comprising recording, in an append-only audit log, one or more of biometric data access events, intervention outputs, confidence scores, supervisory routing events, supervisory approval actions, outcome metrics, or session-specific intervention selection parameter updates.
